(19) United States
(12) Patent Application Publication
(10) Pub. No.: US 2023/0402169 A1
(43) Pub. Date: Dec. 14, 2023

(54) METHOD AND SYSTEM FOR TRACKING MEDICAL TECHNOLOGY PRODUCT AND MEDICAL TECHNOLOGY PRODUCT

(71) Applicant: GREINER BIO-ONE BRASIL SERVICE TECH SISTEMAS, PRODUTOS E SERVIÇOS PARA SAÚDE LTDA, Americana-SP (BR)

(72) Inventor: Haroldo FONTES GRACI, Campinas SP (BR)

(21) Appl. No.: 18/248,975

(22) PCT Filed: Oct. 13, 2021
(86) PCT No.: PCT/BR2021/050444

§ 371 Date: Apr. 13, 2023

(30) Foreign Application Priority Data
Oct. 14, 2020 (BR) BR102020021049 1
Mar. 25, 2021 (BR) BR102021005819 6

(51) Int. Cl. G16H 40/40

(52) U.S. Cl. CPC G16H 40/40

(57) ABSTRACT

A method of tracking medical technology product (100) is described, the medical technology product (1,160) being provided with a product identifier code (2), wherein the method comprises the steps of: linking the product identifier code to a patient's identifier code (A) and generating a plurality of indicators (I_{O}, I_{G}) from the step of linking the product identifier code (2) to a patient's identifier code (4). A system of tracking medical technology product as well as a medical technology product are also described.

The present invention relates to a method and system for tracking medical technology product. More specifically, a methodology and system capable of tracking medical technology product, such as a collection tube used in health care settings, is described.

### DESCRIPTION OF THE STATE OF THE ART

Medical technology products are widely used on a daily basis in various health care settings, such as hospitals, laboratories, veterinary clinics, vaccination centers, among others.

From among the various medical technology products used daily, the use of the so-called collection tubes is highlighted.

Such collection tubes are commonly used to collect a blood sample from a given patient, a sample that will be later analyzed and whose analysis result will be reported to the patient.

In one example, a patient receives a prescription from a medical professional to perform a certain test; thus, the patient goes to a hospital setting (laboratory clinic, hospital, among others) to perform the test through the collection of blood. In certain instances, a health care professional goes to the patient's home (or any other location) to carry out the collection.

Thus, the blood sample is stored in the collection tube, subsequently analyzed in the laboratory and the result reported to the patient.

However, the current state of the art does not disclose an effective means to track collection tubes used in hospital settings.

More specifically, the state of the art does not disclose any means to manage the large amount of collection tubes used in hospital settings, so that, in some instances, expired collection tubes are still kept in use, because there is no way to manage, control and track the use of said tubes in hospital settings.

The present invention describes a methodology and system capable of managing and tracking collection tubes (or any other medical technology product) used in health care settings.

Based on the link between the collection tube and the patient, there are several advantages arising from the present invention, so that, with the proposed methodology, the hospital manager will be aware of which collection tubes are being used for certain tests and where such tests are being performed.

Furthermore, the present invention allows managing which collection tubes have expired, thus allowing the manager to exchange such tubes.

It is noteworthy that the teachings of the present invention are beneficial not only to the hospital manager, but also to the professional who performs the blood collection (health care professional). In one example, the methodology and system proposed in the present invention allow a collection sequence to be indicated to the professional, which sequence aims to inform the professional of the collection order that should be performed for a given patient.

Several advantages arising from the teachings of the present invention will be addressed throughout the following description.

### OBJECTS OF THE INVENTION

The present invention is intended to propose a method of tracking medical technology product used in a health care setting;

The present invention is also intended to propose a method of tracking medical technology product, wherein the method comprises the step of reading a product identifier code of the medical technology product;

One object of the present invention is to propose a method that comprises the step of linking the product identifier code to the patient code;

The present invention is also intended to propose a method of tracking medical technology product that comprises the step of generating a plurality of indicators, wherein the indicators are associated at least with the health care setting operator and the health care setting manager;

The present invention is also intended to propose a method of tracking medical technology product, which comprises the step of comparing a data relative to the expiry date with the current date;

It is an object of the present invention to propose a system for tracking medical technology product;

The present invention is intended to propose a tracking system operable in a first environment and in a second environment;

The present invention is intended to propose a tracking system that is in accordance with the methodology addressed in the present invention.

### BRIEF DESCRIPTION OF THE INVENTION

A method of tracking medical technology product is described, the medical technology product being provided with a product identifier code, wherein the method comprises the steps of: linking the product identifier code to a patient's identifier code, generating a plurality of indicators from the step of linking the hospital material identifier code (product identifier code) to a patient's identifier code.

A system for tracking medical technology product is also described, the system comprising at least one medical technology product provided with an identifier code (product identifier code), a first device able to read the product identifier code, wherein the system comprises means to link the product identifier code to a patient's identifier code.

A non-transitory computer readable medium is also described, the readable medium storing instructions, which instructions when executed by a processor carry out the steps of linking a product identifier code to a patient's identifier code and generating a plurality of indicators from the step of linking the product identifier code to a patient's identifier code.

### BRIEF DESCRIPTION OF THE DRAWINGS

The present invention will now be described in more detail based on one embodiment shown in the drawings. The figures show:

FIG. 1—is a representation of a medical technology product that can be used in accordance with the teachings of the present invention;

FIG. 2—is a block representation of the method proposed in the present invention;

FIG. 3—is a representation of the step of reading a patient's identifier code;

FIG. 4—is a representation of the step of reading a product identifier code;

FIG. 5—is a representation of a step of confirming the link between the medical technology product and the patient;

FIG. 6—is an exemplary illustration of the operator indicator I_{O} (indicator to the operator) displayed on the screen of the first device;

FIG. 7—is an illustration of the representation of the collection sequence displayed on the screen of the first device;

FIG. 8—is an additional illustration of the representation of the collection sequence displayed on the screen of the first device, showing a second step of collection;

FIG. 9—is an additional illustration of the representation of the collection sequence that is displayed on the screen of the first device, showing a third step of collection;

FIG. 10—is an exemplary illustration of the activity report displayed in a second environment;

FIG. 11—is an exemplary illustration of the test panel (exam panel) that is displayed in a second environment;

FIG. 12—is an exemplary illustration of the control panel that is displayed in a second environment;

FIG. 13—is a representation of the step of comparing the data on expiration date Dₑₓₚ of the hospital equipment with the data in the current date D_{CURRENT}, wherein FIGS. 13 (a) and 13 (b) show a compliance scenario and FIG. 13(c) shows a non-compliance scenario;

FIG. 14—is a representation of the step of showing an error message on the first device;

FIG. 15—is a representation of an adhesive means comprising the product identifier code, wherein FIGS. 15 (a) to 15 (e) show that one portion of the adhesive means acts as a criticality classifier.

FIG. 16—is a representation of a medical technology product that can be used in accordance with the teachings of the present invention;

FIG. 17—is a block representation of the some of the steps of the method of the present invention;

### DETAILED DESCRIPTION OF THE FIGURES

The present invention describes a method and system of tracking medical technology product used in a health care setting.

By health care setting is meant any health care facility, such as hospitals, clinics, laboratories, vaccination sites, among others.

The term health care setting can also be interpreted as a nursing home, emergency care units, health centers, emergency treatment centers, vaccination centers, among others. Basically, any facility related to health care should be understood as a health care setting. Also, the health care setting can be interpreted as the home of a specific patient. The term health care setting can be also interpreted as any location where a patient goes to undergo a particular test and/or medical procedure.

In a preferred embodiment, the term health care setting should be interpreted as a hospital or laboratory where a specific patient goes to collect blood and perform a specific test.

Thus, a method of tracking medical technology product used in a health care setting is described.

Medical technology product, on the other hand, should be understood as any material (product) used in the health care setting for which tracking is desired.

In an illustration, said medical technology product can be understood as a vaccine, a collection tube, a prosthesis, a catheter, a needle, a particular package, among others.

In a preferred embodiment, the teachings of the present invention will be described so that the medical technology product is to be understood as a collection tube, such as a blood collection tube, such as those tubes commonly used for collecting a blood sample which will be later analyzed, thus reporting the result of said analysis to the patient. It is noteworthy that reference to blood collection should not be considered a limiting feature of the present invention.

It should be noted that any collection tube can be used in accordance with the teachings of the present invention, regardless of its material of manufacture or shape (conical, round, or any other shape), either sterile or non-sterile tubes used and regardless of the type of lid (closure) used.

Furthermore, tubes of any size can be used in accordance with the teachings of the present invention.

In summary, any component used in a health care setting and able to store a sample could be used in accordance with the teachings of the present invention.

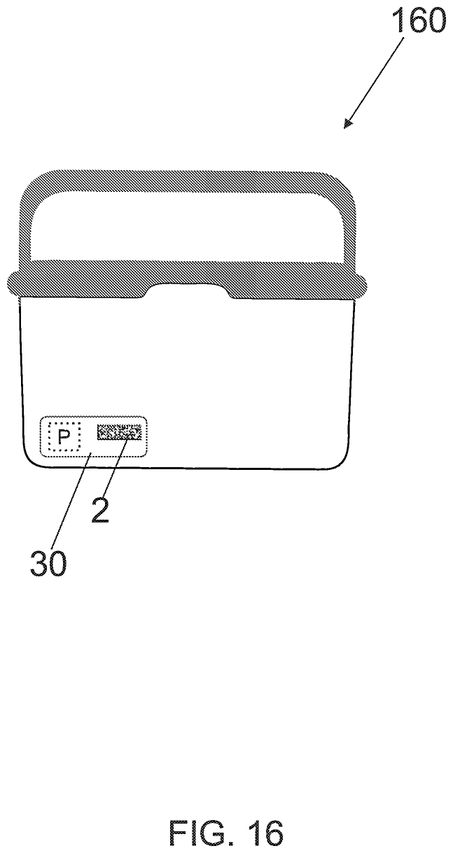

Additionally, the term medical technology product 1 may for example be understood as a package that stores a set of products, such as a package 160 that stores a certain number of units of a specific product, for example, collection tubes. Reference is made to FIG. 16. But the term medical technology product 1 may for example also be understood as a product comprised in the package and/or a one uniquely identified product.

Thus, said package 160 can be tracked, for example, from an entity that sells the tubes to a purchasing entity. It is proposed that a product identifier code 2 may be associated with package 160 and/or each of the products comprised therein and/or a one uniquely identified product 160.

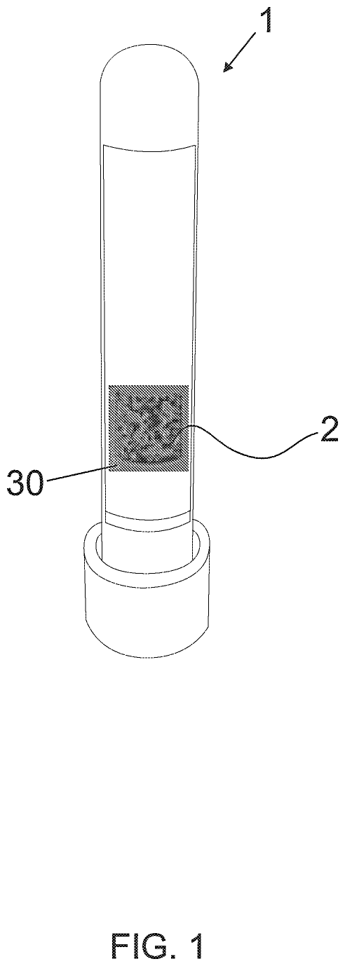

FIG. 1 is a representation of a medical technology product 1 that can be used in accordance with the teachings of the present invention. The medical technology product 1 illustrated in the present figure is a collection tube, such as a blood collection tube.

Billions of collection tubes are manufactured annually and purchased by hospital managers.

A given health care setting can purchase a huge amount of these tubes (on the order of millions) and thus distribute them among its health care units so that blood collection can be carried out. For example, a certain health care setting, such as a clinical laboratory, may have 500 health care units across a certain region. Thus, the manager of said health care setting is responsible for purchasing the medical technology product (collection tubes) and thus distributing them to the 500 units.

The teachings of the present invention propose that the medical technology product 1 comprises a product identifier code 2. Thus, and in reference to FIG. 1, a product identifier code 2 is present. In one embodiment, the product identifier code 2 may for example be understood as a printed number and/or an optically, magnetically or electronically readable code, i.e., 1D, 2D, QR code, magnetic code, electromagnetic code, RFID, NFC or any other code known in the state of the art. It is thus understood that the methodology proposed in the present invention teaches the step of associating an identifier code 2 (product identifier code) with a medical technology product 1,160. The product identifier code may be provided on a label placed on a medical technology product or its package.

In one embodiment, said identifier code 2 must be placed on a surface of the medical technology product 1, so that any surface of the product 1 can receive said identifier code 2.

In this embodiment where the medical technology product 1 is understood as a collection tube, it is proposed that the identifier code 2 be placed on any portion of the tube. Placement of the identifier code 2 on the tube cap is also acceptable.

In one embodiment, said identifier code 2 can be associated with the medical technology product 1 through an adhesive means 30. Thus, the adhesive means 30 comprising the identifier code 2 is associated with the medical technology product 1. The adhesive means 30 can be understood as a label, an adhesive, a stamp, a plate, or any other equivalent element.

The adhesive means 30 can be of any size, as long as it allows association with the medical technology product 1,160. Furthermore, the adhesive means 30 may comprise not only the patient's identifier code 2, as well as additional information, such as the manufacturer's name of the medical technology product as well as any additional information that the manufacturer and/or recipient of the medical technology product 1 deems appropriate.

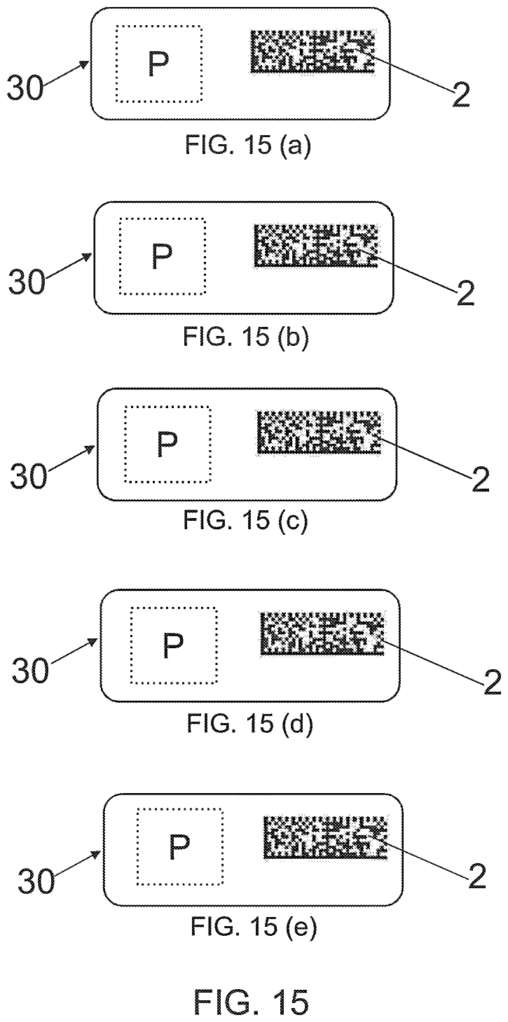

Also, and with reference to FIG. 15, the adhesive means 30 can comprise at least one portion P which acts as a criticality classifier. By criticality classifier is meant that at least one portion (part) of the adhesive means 30 will indicate at least one criticality level of said medical technology product 1 and, accordingly, of the patient associated with the product 1.

In a valid embodiment, such criticality classification is made using colors, thus, portion P of the adhesive means 30 can have a certain color, said color showing to the operator or manager of the health care setting a criticality degree of the medical technology product 1 and accordingly of the patient.

Thus, portion P being of a first color can indicate a classification in which the patient has a simple disease and is under observation.

Portion P being of a second color, on the other hand, can indicate a classification in which the patient has more than one disease with possible systemic impairments.

Portion P being of a third color can indicate the classification where the patient has a systemic impairment with possible evolution to a critical condition.

Moreover, portion P being of a fourth color can indicate a classification where the patient is in critical state.

Moreover, portion P being of a fifth color can indicate a classification in which the patient has an irreversible disease.

In one example, the first, second, third, fourth and fifth colors could be established, respectively, as the colors: green, orange, yellow, red and black.

Obviously, such color description should not be considered as a limiting feature of the present invention, so any color as well as any classification pattern can be used. A classification pattern is understood as, for example, a patient having an irreversible disease.

It is noteworthy that portion P of the adhesive means 30 can be of any size and the size shown in FIG. 15 should not be regarded as a limitation of the present invention.

Furthermore, association of the identifier code 2 with the material 1,160 through an adhesive means 30 does not represent a limiting feature of the present invention, so that any form of association would be acceptable.

The product identifier code 2 can store certain information (data) on the medical technology product 1,160. In one embodiment, the product identifier code 2 stores at least one of: a medical technology product description data D_{DESCR}, a medical technology product batch data and data on the medical technology product expiration date D_{EXP}.

By medical technology product description data D_{DESCR} is meant that the reading of the identifier code 2 will provide information on the description of the product. As an example, the medical technology product description describes that the product is a “100 milliliter collection tube with a screw cap”.

In summary, the description data can be understood as data describing the medical technology product, allowing the reader (user) of the aforementioned description data D_{DESCR} to know which product 1 is being referred to.

In an equally valid example, the description data can describe that the medical technology product is a “SARS-CoV-2 vaccine”. Obviously, reference to SARS-CoV-2 should be considered as an exemplification, without being a limitation to the teachings proposed herein.

Further, the medical technology product description data D_{DESCR} may describe that the product refers to a set of products, thus, the description data may indicate that the product 1 refers to “300 units of needles a specific type”. Such a description is usually found in embodiments where the product identifier code 2 is placed on a package comprising a plurality of medical technology products, as illustrated in FIG. 16.

On the other hand, medical technology product batch data should be understood as information (record) regarding the manufacturing batch of the product and/or the set of products. Therefore, the form in which such batch data is presented must be determined by the product manufacturer.

In an equally valid embodiment, the medical technology product batch data may contain information on the manufacture batch of a set of products, for example: “300 units of needles from batch XYZ”.

Data on expiration date D_{EXP} should be understood as the reading of identifier code 2 will provide the expiration date D_{EXP} of the medical technology product 1. Thus, information (data) referring to the expiration date D_{EXP} of the product is stored in identifier code 2.

In an equally valid embodiment, data on expiration date Dₑₓₚ may contain information on the expiration date of a set of medical technology products, for example: “300 units of needles expiring on Jan. 1, 2022”.

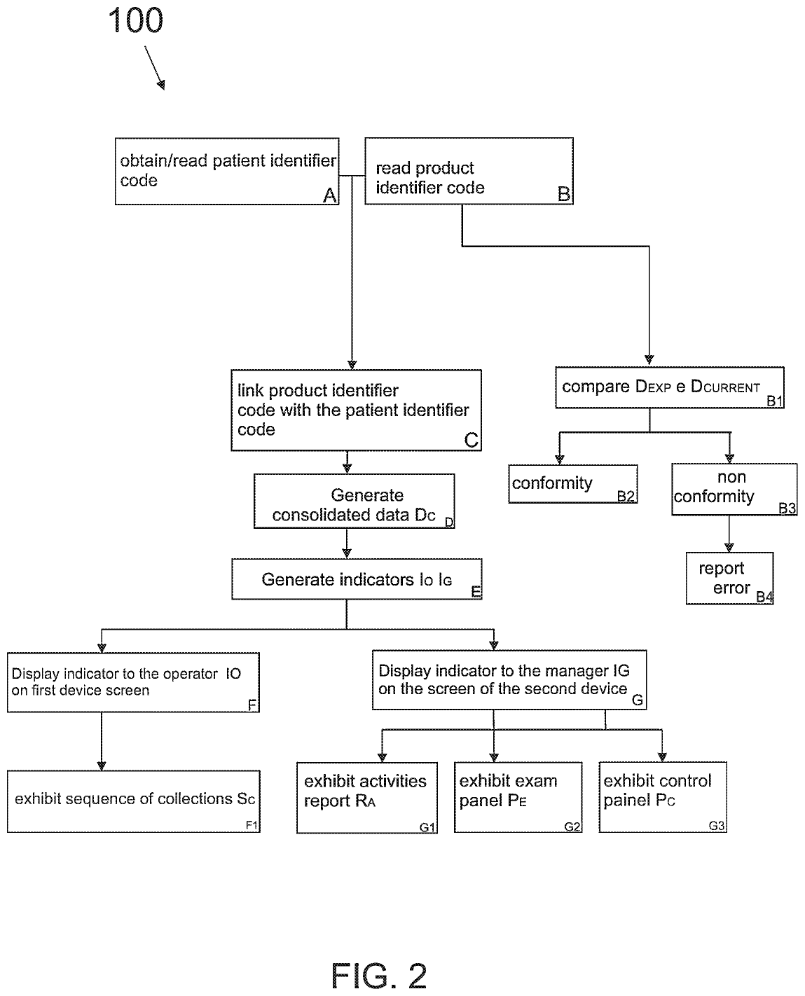

FIG. 2 is a block illustration of the method of tracking a medical technology product 100 as proposed in the present invention.

The method 100 proposed in the present invention comprises the step of linking the product identifier code to a patient's identifier code. This step is illustrated in FIG. 2 through block C.

More specifically, the step of linking such codes can be carried out to associate the medical technology product 1 with the patient; in other words, such an association will determine that the given medical technology product 1 was used for the collection of blood from a certain patient, which patient underwent a certain test, in a certain location and at a certain time.

For such a link to be made, a patient's identifier code 4 must be generated by the health care setting. In this sense, prior to performing a test and/or procedure patients are known to be registered in the health care settings, thus generating an identifier code.

In this sense, the patient's identifier code 4 comprises basic data on the patient, such as full name, age, weight, height. Furthermore, the patient's identifier code 4 further comprises data regarding which tests the patient will perform (or has performed) in the health care setting, thus establishing a test data D_{TEST}.

In one embodiment, the patient's identifier code 4 can be configured as a bar code (optically readable code) inserted into a patient's registration form, so that said registration form can be printed by a hospital operator or can be digitally stored in the hospital database. Any optically readable code known in the prior art could be used (1D, 2D, QR, among others). Additionally, or alternatively, the patient's identifier code 4 can be arranged on a bracelet, badge (or any other equivalent element) that is worn by the patient. Furthermore, the patient's identifier code 4 can be arranged on a tag that is associated (in any way) with the patient. It is thus understood that said label can be configured as the adhesive means 30, which comprises the criticality classifier.

In an equally valid embodiment, the patient's identifier code 4 can be configured as a sequence of numbers that is linked to a certain patient.

Thus, the linking step between the product identifier code and the patient's identifier code (step C) begins by obtaining the patient's identifier code 4 in the health care setting, as represented by block A in FIG. 2.

In one embodiment, the patient's identifier code 4 is configured as a bar code displayed on a patient registration form. Thus, the step of linking the product identifier code and the patient's identifier code C further comprises the step of reading the patient's identifier code 4.

In particular, said reading step must be performed by an operator in the health care setting, for example, the operator responsible for carrying out the test on the patient, so that, in the case of a blood collection, the operator is the one responsible for collecting blood from the patient. More specifically, the reading of the patient's identifier code 4 is carried out by a first device 10 of the health care setting operator.

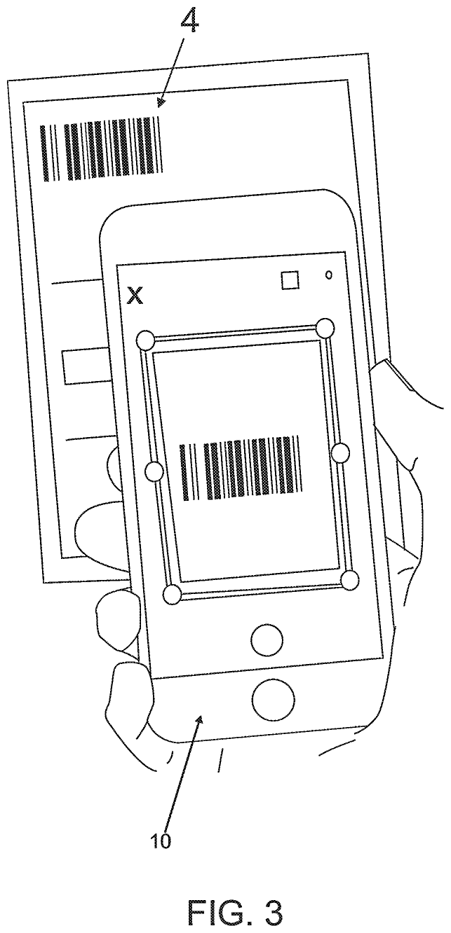

In a valid embodiment, said first device 10 is configured as a cell phone, as illustrated in FIG. 3. Thus, the health care setting operator must, using the cell phone 10 optical instrument (camera), read the patient's identifier code 4, as shown in FIG. 3. Once said reading is carried out, the patient's identifier code is stored, registered and processed, for example, in the operator's cell phone 10 or in the cloud. It is noteworthy that the optical instrument can be configured as the camera of the cell phone itself or can also be configured as a camera associated with the cell phone through a connection means, such as a USB cable, bluetooth, or equivalent means.

When reading the patient's identifier code 4, a first sound signal is emitted by the first device 10, thus providing an audible means of confirmation that the reading of the patient's identifier code has been performed. Additionally, or alternatively, a visual signal could also be displayed on the screen of the first device 10, thus confirming that the reading has taken place.

In one embodiment where the patient's identifier code 4 is stored in a database in the health care setting, it is the operator's responsibility to identify the patient's identifier 4 and thus proceed with the proposed methodology. The manner by which such a code is obtained by the operator does not represent the essential aspect of the present invention. Once the patient's identifier code 4 has been identified and obtained, the first sound signal will be issued to the operator.

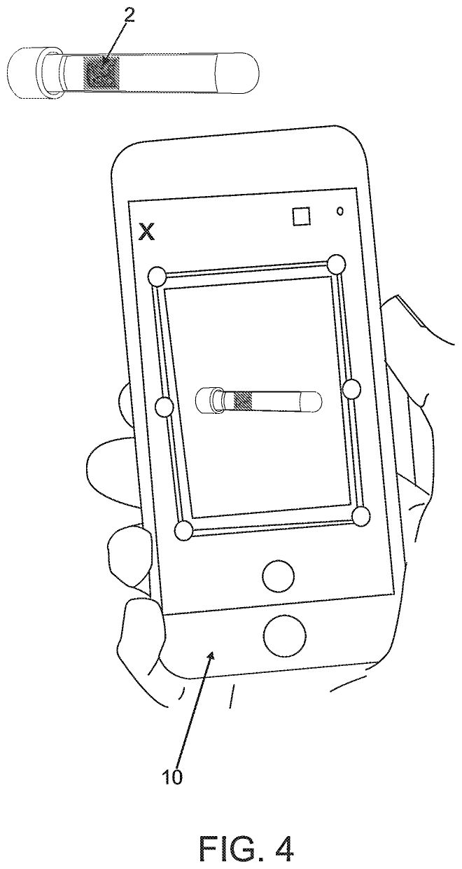

Once the patient's identifier code 4 has been obtained, the methodology proposed in the present invention teaches that the operator of the health care setting must read the product identifier code 2, as represented by block B of FIG. 2. Thus, the operator must, using the first device 10, read the code 2 placed on the collection tube 1, as shown in FIG. 4. Once said reading is carried out, the product identifier 2 code is stored, registered and processed, for example, in the operator's cell phone 10 or in the cloud.

Moreover, when the product identifier code 2 is read, the first sound signal can also be emitted, thus enabling the operator to receive sound confirmation that the reading has been performed.

Further, a visual confirmation could also be displayed on the screen of the first device 10, thus visually confirming that the reading has been performed. The proposal to carry out the sound and/or visual confirmation are perfectly valid.

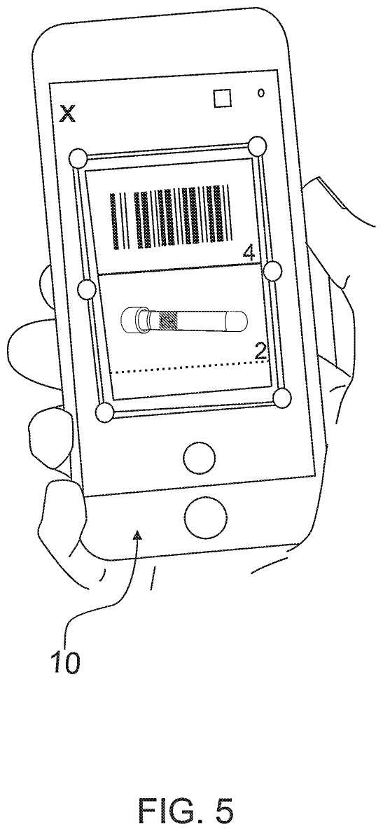

Once the product identifier code 2 has been read and the patient's identifier code 4 has already been identified, a confirmation step can be requested from the hospital operator, as shown in FIG. 5. In the aforementioned confirmation step, two codes 2 and 4 can be displayed on the screen of the first device 10 and, after confirmation by the operator, the step of linking the product identifier code to the patient's identifier code will have been completed.

It is proposed that, when generating the link between the identification code of hospital equipment 2 and the identification code of patient 4, the first sound signal is also emitted, thus confirming to the operator that the link between hospital equipment and patient has been performed.

The link between the product identifier code 2 and the patient's identifier code 4 generates consolidated data D_{C} formed by the set of information contained in the product identifier code 2 and also contained in the patient's identifier code 4. The step of generating a consolidated data D_{C} is illustrated by block D in FIG. 2.

Thus, the consolidated data D_{C} is set as data that can be read by a processor and that comprises at least information on: description of the medical technology product D_{DESCR}, the product batch and the product expiration date D_{EXP}. The consolidated data D_{C} can also comprise information on the place and date when the link between the product and the patient's identifier code was generated, that is, thus comprising information on the place and date of blood collection. The consolidated data D_{C} further comprises information on the patient, which information has been previously stored in the patient's identifier code 4. Thus, the consolidated data D_{C} comprises information such as: full name, age, weight, patient's height and which tests the patient will perform (or has performed) in the health care setting. It is proposed that the consolidated data D_{C} can also include information about the patient's identifier code 4 and the product identifier code 2. It is understood that the consolidated data D_{C} is data that can be read, registered and processed by a microcontroller/processor. Also, the consolidated data D_{C} can be stored in a memory and transmitted between electronic devices through a network protocol or any protocol that allows one to send/receive data.

The place of location of the processor that is able to read, send/receive the consolidated data D_{C} is not an essential feature of the present invention, so any location and form of arrangement would be valid. It is understood that cloud processing is perfectly applicable.

Therefore, the consolidated data D_{C} also stores information on the patient's identifier code 4 and the product identifier code 2.

The method of tracking medical technology product 100 as proposed in the present invention further comprises the step of generating a plurality of indicators from the step of linking the product identifier code to the patient's identifier code. Said step is illustrated in FIG. 2 through block E.

Generation of such indicators allows the medical technology product 1 to be tracked, thus providing better control and management of all products existing in a health care setting or in a network of health care settings.

More specifically, such indicators are associated with at least one of: a health care setting operator I_{O} and a health care setting manager I_{G}.

By health care setting operator is meant the health care setting professional who collects the patient's blood, thus the health care setting operator is understood as the professional who works in the laboratory/hospital in which the patient performs a certain test and/or procedure. It is noteworthy that the test does not necessarily have to be performed in the health care setting, so that collection can be performed at any location, for example, in the patient's home or in a place determined by the patient or by the management of the health care setting.

Furthermore, the health care setting operator can also be understood as the operator who receives a certain medical technology product, such as the operator who receives a set of products from a certain point of origin.

The health care setting manager, on the other hand, must be understood as the management of the health care setting, which manager is responsible for the administration of at least one health care setting unit.

Thus, the health care setting manager can be responsible for managing several health care setting units, such as units spread over a particular city, region or country.

Finally, a health care setting patient is understood as a patient who goes to a health care setting (or to any location) to undergo a certain test, which test will require the use of a medical technology product 1 to be handled by the health care setting operator. The teachings of the present invention are also beneficial to the patient, since, by ensuring full traceability of all medical technology products used in the testing process, the present invention allows that, in the event of any failure with one of the medical technology products, the responsible person can determine which patients were affected and hence request a new collection or test.

It is thus understood that the teachings of the present invention are beneficial not only to the patient, but also to the employee (operator) and manager of the health care setting.

Thus, the methodology proposed in the present invention teaches that the plurality of indicators comprises at least one indicator to the health care setting operator I_{O}, wherein the indicator to the health care setting operator is displayed to the health care setting operator. Said step is illustrated in block F of FIG. 2.

Thus, and based on the previously generated consolidated data D_{C}, the indicator to the health care setting operator I_{O} correlates (associates) a plurality of medical technology products with a plurality of users (patients) of the health care setting.

Thus, the indicator to the health care setting operator I_{O} can be understood as a report that displays information about the consolidated data D_{C}, thus generating a consolidated data D_{C} report.

Displayed on the screen of the first device 10, the operator indicator I_{O} can display information on all links between materials 1 and patients performed on a specific day or period.

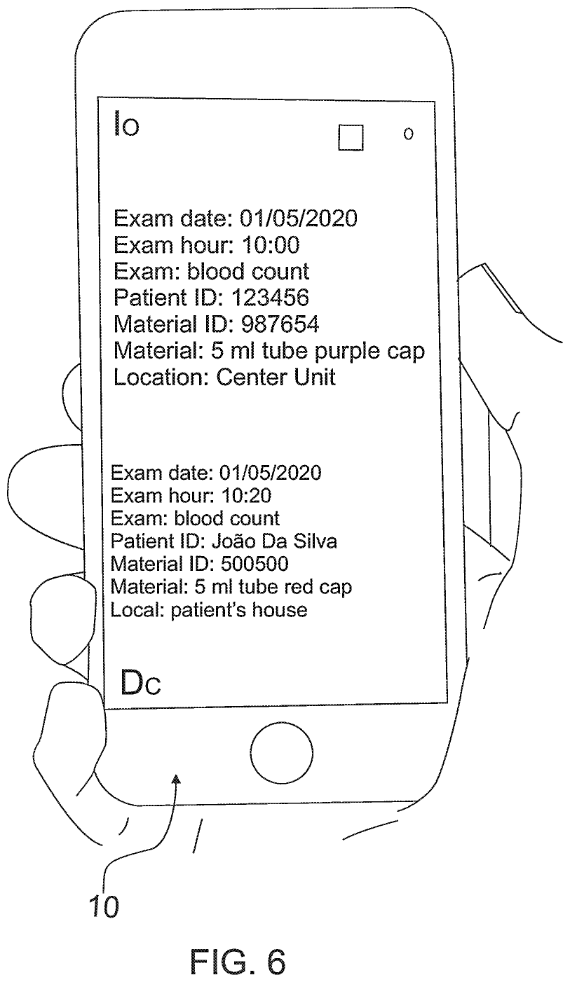

FIG. 6 is an exemplary illustration of the operator indicator I_{O} displayed on the screen of the first device 10. The indication and visualization of consolidated data D_{C} obtained from the link previously established between the medical technology product 1 and the patient is seen.

Thus, a first consolidated data D_{C} can inform the health care setting operator of the date and time when the collection was performed. Also, the operator can view a consolidated data D_{C} regarding which test is to be performed (or which has already been performed).

Also, consolidated data D_{C} relative to the patient's identifier code (patient ID) and the product identifier code (material ID) can be displayed, as well as a description D_{DESCR} of the hospital material (product) that is (or was) used on the test.

Consolidated data D_{C} relative to the patient's name, age, sex and height can also be displayed via the operator indicator I_{O}. Furthermore, the operator indicator I_{O} can also show the location where the collection will be carried out (or was carried out), as shown in FIG. 6.

In one embodiment, the health care setting operator indicator I_{O} can display a sequence of S_{C} collections to the laboratory personnel. Thus, it is understood that the methodology proposed in the present invention further comprises the step of generating and displaying a sequence of collections S_{C} to the health care setting operator, wherein the sequence of collections S_{C} is displayed on the first device 10. Said step is represented in block μl of FIG. 2.

From the link previously made between the product identifier code 2 and the patient's identifier code 4, the collection sequence S_{C} is generated from an association between the medical technology product description data D_{DESCR} and the patient's identifier code 4. More specifically, the collection sequence S_{C} is generated from an association between the medical technology product description data D_{DESCR} and test data D_{TEST}.

Thus, the step of generating the collection sequence S_{C} further comprises the step of obtaining information (data) concerning the medical technology product description D_{DESCR} and obtaining information (data) on the test data D_{TEST}.

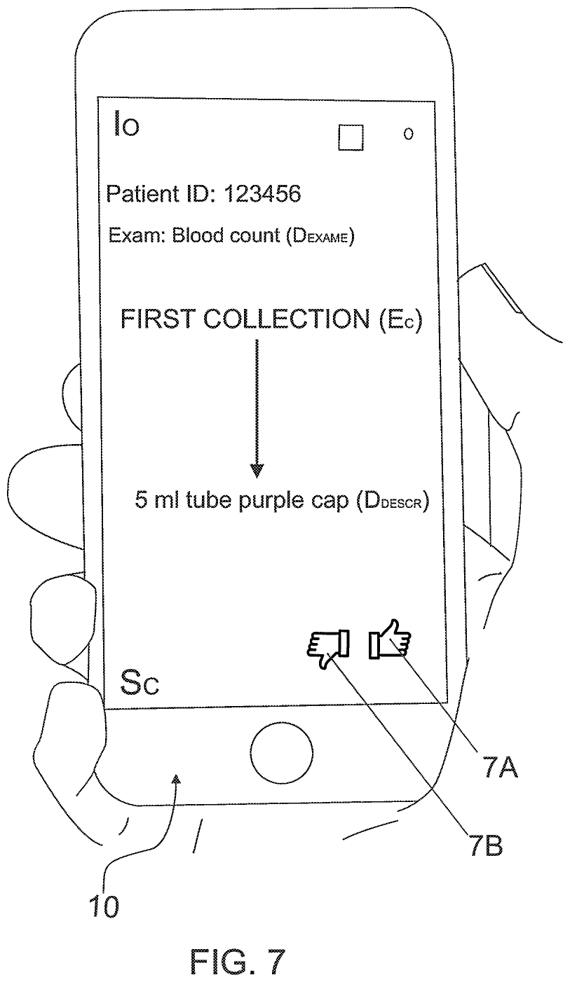

Thus, the collection sequence S_{C} is displayed on the first device 10 of the health care setting operator, as depicted in FIGS. 7, 8 and 9.

By collection sequence S_{C} is meant the indication to the operator of a correct sequence of medical materials 1 to be used for collecting blood for a particular test. Wherein said indication is displayed on the screen of the first operator device 10.

In this sense, depending on the test (medical exam) to be performed it is known that more than one collection tube (medical technology product) has to be used; also, a given test may require the use of a specific sequence of collection tubes, so that, if this sequence is not followed, the test and indication of the result to the patient may be compromised, for example, showing an inconclusive result or even a false positive or false negative.

Thus, and with reference to FIGS. 7 to 9, the collection sequence S_{C} shows on the display 10 screen, test data D_{TEST} as well as data on the description of the medical technology product D_{DESCR}. Thus, it is understood that the collection sequence S_{C} indicates on the device screen 10, which test that particular patient will make (Dₜₑₛₜ) and the sequence of collection tubes that should be used for the test.

In a valid embodiment, it is proposed that the sequence of collection tubes be indicated on the device 10 screen and through an indication relative to which medical technology product should be used in a first collection step E_{C} (first step), as represented in FIG. 7.

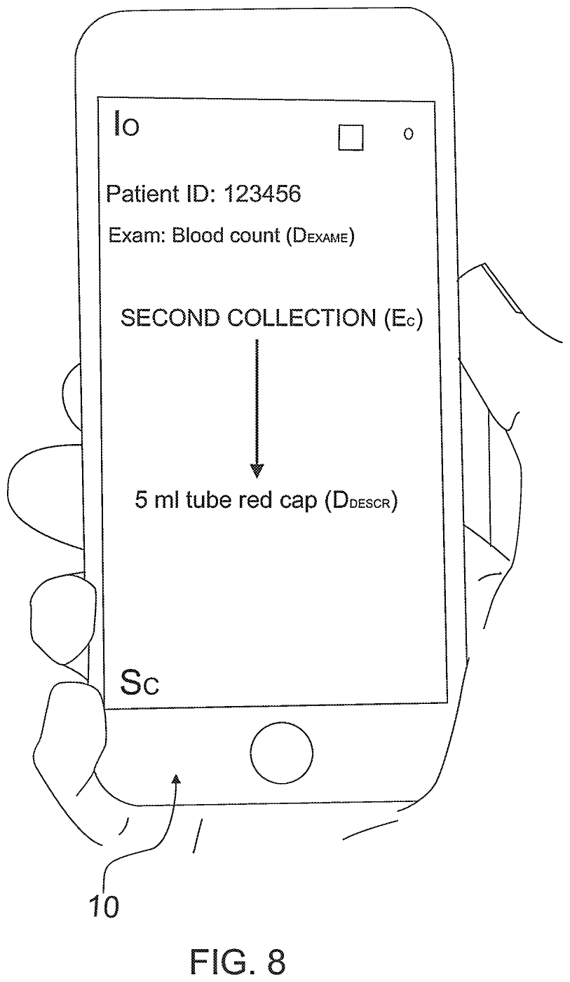

Subsequently, the collection sequence S_{C} illustrates to the operator a second collection step E_{C} that must use a specific medical technology product, as represented in FIG. 8.

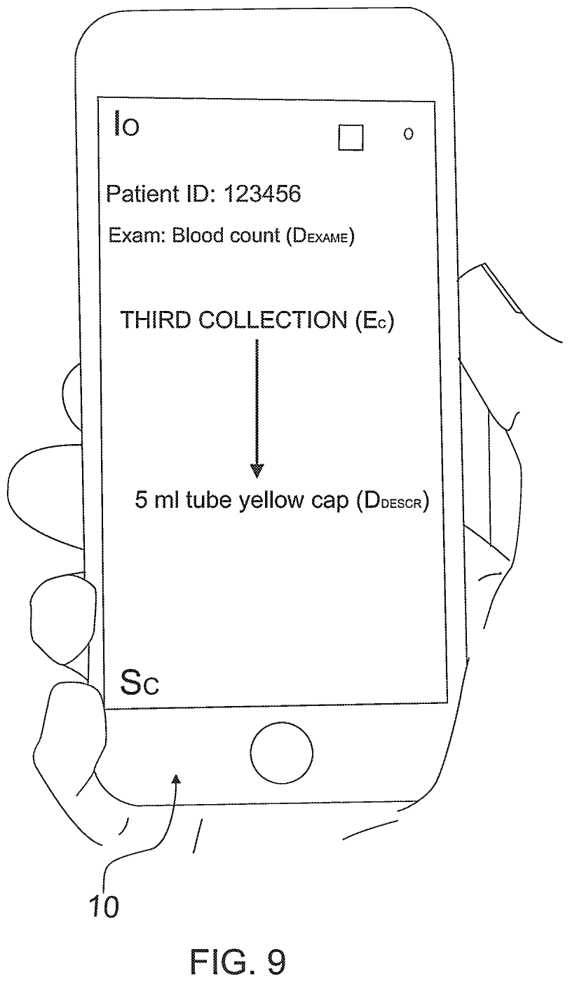

Finally, an exemplification of collection sequence S_{C} shows the operator the next collection step E_{C} (third sample) as well as the description of the medical technology product D_{DESCR} to be used in this step, as illustrated in FIG. 9.

Thus, the collection sequence S_{C} illustrates at least the test data D_{TEST}, the medical technology product description data D_{DESCR} and a collection step E_{C}, as depicted in FIGS. 7, 8 and 9. In one valid embodiment, the collection sequence S_{C} can further illustrate the patient's identifier code 4, either by means of a numeric code through the patient's name, or by any other means that enables the patient to be identified.

Obviously, the form of representation of collection sequence S_{C} as illustrated in FIGS. 7, 8 and 9 should be considered as an illustrative representation only, that is, the representation illustrated in the figures should not be considered a limiting feature of the teachings proposed herein.

Also, description of the collection sequence S_{C} comprising three collection steps E_{C} should also not be considered a limiting feature, so any number of collection steps E_{C} can be used. Thus, the collection sequence S_{C} could only indicate to the operator a collection step and E_{C}, or any other number of collection steps E_{C}, depending only on the test to be performed.

In one embodiment, the collection steps E_{C} of a given test can be previously stored in a database.

Furthermore, and in reference to FIG. 7, after each of the collection steps has been performed, a confirmation step can be displayed to the user, so that, with a positive confirmation 7A (the step was properly performed), the next collection step will be displayed on the first device 10. If the operator indicates a negative confirmation 7B (the step was not correctly performed), said step must be performed again.

It is noteworthy that said confirmation step (positive or negative) may also be associated with an audible indication, thus emitting a sound signal to the operator.

Thus, with a positive confirmation, the first sound signal must be emitted to the user, whereas in the case of a negative confirmation, a second sound signal must be emitted to the user, so that the second sound signal emits a different sound than the first sound signal.

As an example, a negative confirmation 7B could be generated due to a defect in the collection tube or even due to the fact that the patient is not able to perform the procedure.

In this sense, in the case of a defect in a collection tube, the proposed methodology teaches that the operator may replace the defective tube with a suitable one, also allowing the storage of data present in the tube identifier code 2 either from the defective tube or the fresh one.

In addition to the indicator to the health care setting operator I_{O}, the methodology proposed in the present invention teaches that the plurality of indicators further comprises at least one indicator to the health care setting manager I_{G}. As discussed above, this indicator is displayed to the health care setting manager. Said step is represented in block G of FIG. 2.

In one embodiment, it is proposed that the indicator to the health care setting manager I_{G} be displayed in a second environment.

Thus, and as previously described, the indicator to the health care setting operator I_{O} can be displayed in a first environment, wherein by first environment is meant the first device 10, such as a cell phone.

The indicator to the health care setting manager I_{G}, in turn, must be displayed on a second environment 20, wherein by second environment 20 is meant a second device operable by the health care setting manager.

Thus, the second environment 20 is preferably set as a web platform that can be accessed by the health care setting manager. In one embodiment, the manager accesses said web platform using an electronic device, such as a computer, tablet, cell phone (or any other equivalent means) and using a login and password.

More specifically, the second environment 20 can be configured so as to receive the links previously generated by the health care setting operator and related to the product identifier code 2 and also related to the patient's identifier code 4.

Thus, each link generated by the operator will be sent to the health care setting manager. Thus, each link generated by the professional responsible for the collection will be sent to the health care setting manager.

Thus, both the health care setting operator and manager are associated with the previously established link.

Thus, and as described above, the link between the product identifier code 2 and the patient's identifier code 4 generates the consolidated data D_{C}, thus, the present invention comprises the step of sending the consolidated data D_{C} to the health care setting manager. More specifically, the consolidated data D_{C} is processed and sent to the second environment 20, which second environment 20 is operable by the health care setting manager.

In one embodiment, the consolidated data D_{C} is sent to the health care setting manager by the health care setting operator using the first device To this end, it is proposed that the first device 10 comprises a suitable processor and memory to store consolidated data D_{C} and then send them to the health care setting manager.

In one embodiment, consolidated data D_{C} is sent to the health care setting manager by the operator for each link made, or, alternatively, it can be sent in blocks, for example, for every 10 links generated. Periodicity of sending the consolidated data D_{C} to the health care setting manager is not an essential aspect of the present invention, so any periodicity would be acceptable.

In a fully valid embodiment, the sending of consolidated data D_{C} to the health care setting manager can be automatically performed by the first device 10, not requiring any interference by the operator.

Thus, the consolidated data D_{C} will be sent to the health care setting manager, and more specifically, the consolidated data D_{C} will be sent to the second environment 20 operated by the health care setting manager.

Accordingly, from the consolidated data D_{C} sent to the second environment, the health care setting manager can generate an activity report R_{A}, which will include information about: (i) the health care setting unit where the link was generated, that is, the place where the link was generated, (ii) the date when the link was generated, that is, the date the test (collection) was performed, (iii) the patient's identifier code 4, (iv) the medical technology product description data D_{DESCR}, (v) of the test data D_{TEST} (vi) an identifier code of the operator responsible for the collection (hence allowing the operations performed by the operator to be tracked), which code is provided by the health care setting. The step of generating and displaying the activity report is represented in block G1 of FIG. 2. Data referring to the location of the health care setting unit (place where the collection was carried out) can be generated through a geolocation service of the first device 10 or any equivalent means. Such location data can be present in the consolidated data D_{C}.

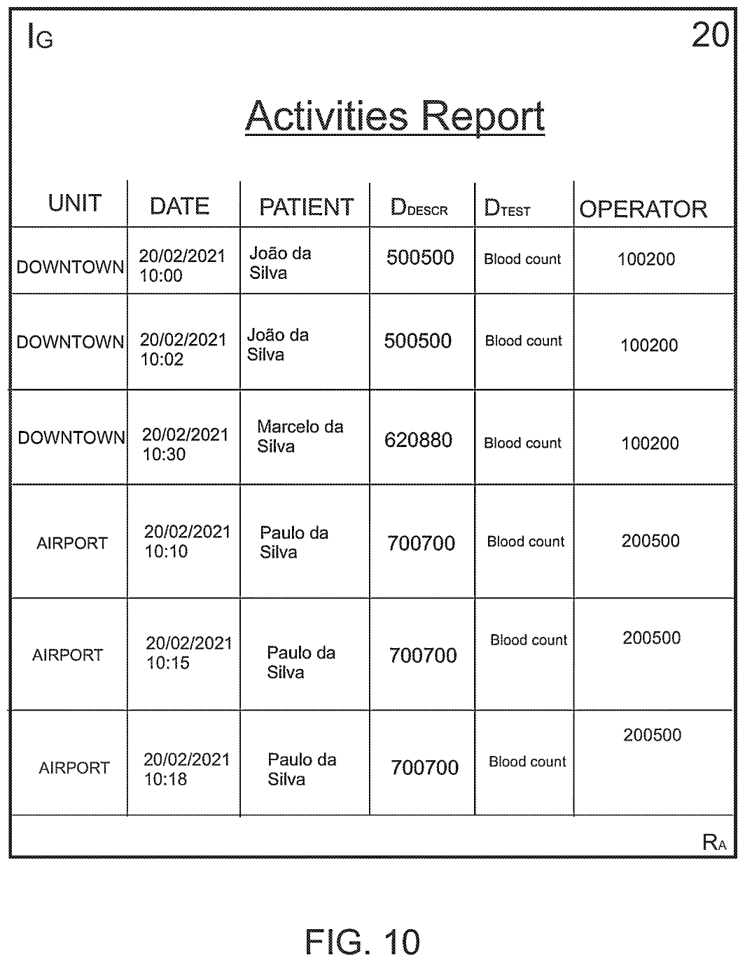

FIG. 10 is an example of the activity report R_{A} generated from the consolidated data D_{C}, wherein said activity report R_{A} is displayed on a screen of the second environment 20, such as on a computer or tablet screen.

Thus, based on the activity report R_{A}, the health care setting manager can, for example, generate a CSV report or even perform the desired classifications and filters. It is noteworthy that the activity report R_{A} can be generated to comprise any of the information present in the product identifier code 2 as well as in the patient's identifier code 4.

Thus, and based on the previous description as well as the content of FIG. 10, it is understood that the activity report R_{A} correlates at least data referring to the health care setting unit in which the link was generated, data referring to the date when the link was generated, data referring to the patient's identifier code, medical technology product description data D_{DESCR} and test data D_{TEST} referring to an identifier code of the operator responsible for the collection.

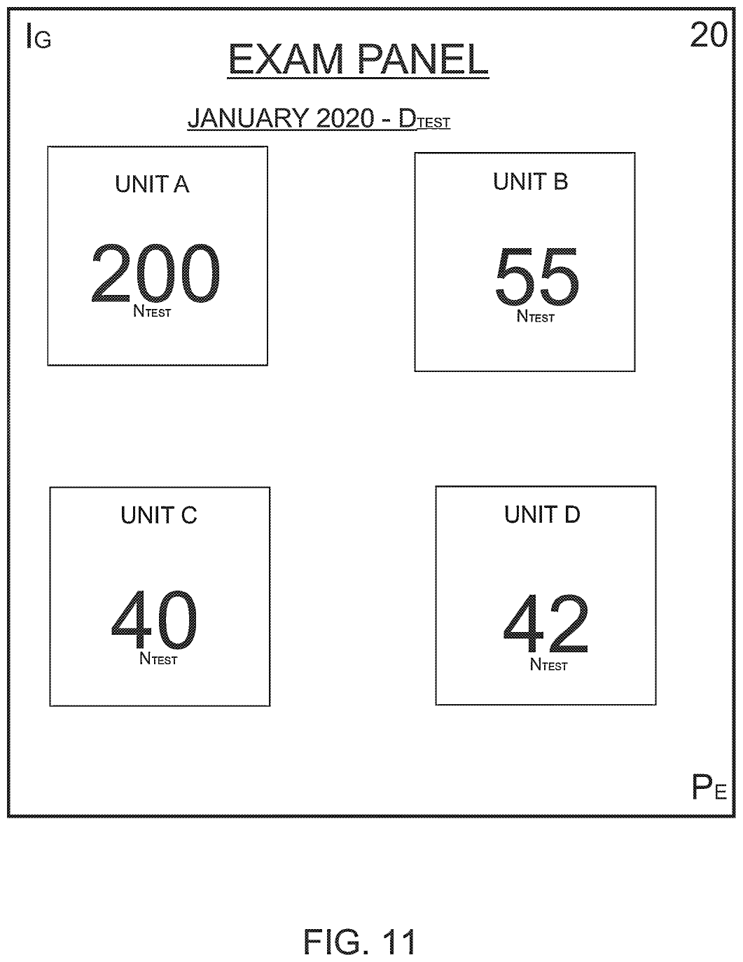

Also, from each of the generated links, the health care setting manager can generate a test panel P_{E} (exam panel) to be displayed on the screen of the second environment 20, as illustrated in FIG. 11 and represented by block G2 in FIG. 2.

By test panel P_{E} is meant the display, on the screen of the second environment 20, of the number of performances of a given test for each of the units under the management of the health care setting manager and considering a period of time to be determined by the manager.

Thus, and referring to the example in FIG. 11, the test panel P_{E} can illustrate (i) data referring to the health care setting unit where the link was generated, such as units A, B, C and D, data (ii) referring to test D_{TEST} and (iii) a data referring to the number of test performed for that unit, which data is referred to as N_{TEST}. It is understood that the test panel P_{E} correlates data referring to the health care setting unit where the link was generated, data referring to the test performed D_{TEST} and data referring to the number of tests performed for that unit N_{TEST}, considering a specific period of time.

Obviously, the illustration of FIG. 11 is merely exemplary, so that any of the data illustrated in such figure should not be considered as a limitation of the present invention.

Also, the illustration of only 4 units is also a mere illustration, as it is common for a certain manager to manage hundreds of units.

In summary, the number of units should not be considered as a limitation of the present invention.

Thus, generation of the test panel P_{E} on the screen of the second device 20 is extremely beneficial to the health care setting manager, thus enabling him/her to manage the replenishment and stock of a certain medical technology product 1 to be used in large quantities for carrying out a particular test.

Furthermore, generation of the test panel P_{E} also allows one to address public health policies by verifying, for example, that unit A has performed an excessive amount of a given test, which fact that may indicate that a certain virus or bacterium is proliferating in the region where unit A is located.

Additionally, the test panel P_{E} can be updated as new links between the medical technology product 1 and the patient are generated by the operator.

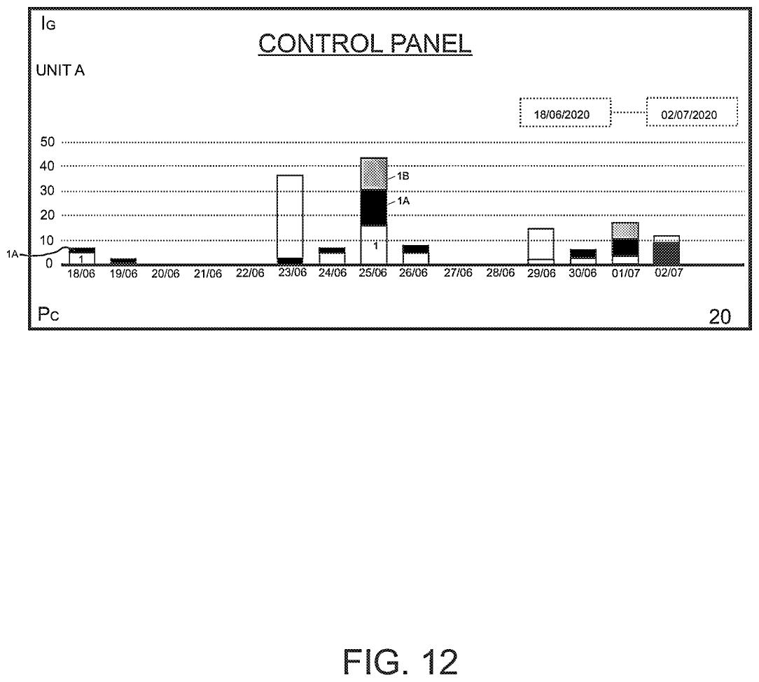

The indicator to the health care setting manager I_{G} can also be configured as a control panel P_{C}, as displayed in FIG. 12. To be displayed on the screen of the second environment 20, the control panel P_{C} is preferably set as a graphical illustration where the horizontal axis shows a specific time period selected by the health care setting manager, wherein said time period it is preferably segmented into days. Block G3 of FIG. 2 shows a step of displaying the control panel P_{C}.

On a vertical axis of the graphic illustration that is part of the control panel P_{C} are arranged the numbers indicating the number of links generated on a given day.

Thus, and preferably using a column indicator, there displayed to the health care setting manager how many links between the medical technology product and the patient were generated in a given day. It can consider a single unit, as shown in FIG. 12, or it can even consider all units under the management of a certain manager.

Also, the control panel P_{C} can also be segmented by the type of medical technology product, i.e. a column indicator for a particular day can be segmented according to the medical technology product description D_{DESCR}. Thus, in one example based on FIG. 12, the column indicator of the date 06/18 can be segmented into a first medical technology product 1 and a second medical technology product 1A.

Similarly, and now considering date 25/06 (June 25), the column indicator can be segmented into the first medical technology product 1, the second medical technology product 1A and even the third medical technology product 1B.

In one example, the first medical technology product 1 can be a “5 ml tube with a purple cap” and the second medical technology product 1A can be a “5 ml tube with a yellow cap” and the third product 1B can be a “tube for capillary blood collection”. It is proposed that each material be indicated on the control panel by a different color pattern.

Thus, based on the previously made description, it is understood that the control panel P_{C} is configured as a graphical indicator that correlates a specific period of time (preferably segmented into days) with the number of generated links, while still allowing segmentation by the type of medical technology product, as shown in FIG. 12.

Thus, the indicator to the health care setting manager I_{G} allows for consultation of graphics and information on the routine and collection process, as well as information on registered units and registered operators. The indicator to the manager I_{G} enables a real-time monitoring of activities and consumption of inputs. In summary, control through the indicator to the manager I_{G} can take place through information/reports that indicate: the use of medical technology products (by day and hour and by laboratory), tests by laboratory, target tube number (by laboratory), recent activities, registered operators, average test time.

The link between the medical technology product 1 and the patient also allows an evaluation to be made on whether a certain medical technology product is within its expiry date.

To this end, the methodology proposed in the present invention teaches that the step of reading the identifier code of the medical technology product further comprises the step of comparing the expiration date data D_{EXP} with a current date D_{CURRENT}, as represented in block B1 of FIG. 2.

As previously described, data referring to the expiration date D_{EXP} of the medical technology product is stored in the identifier code 2, whereas the data referring to the current date D_{CURRENT} can be directly obtained by the date/time system of the first device 10 or even obtained in any way known in the state of the art.

Thus, reading of the product identifier code further comprises the comparison between the data on the expiration date D_{EXP} and data referring to the current date D_{CURRENT}.

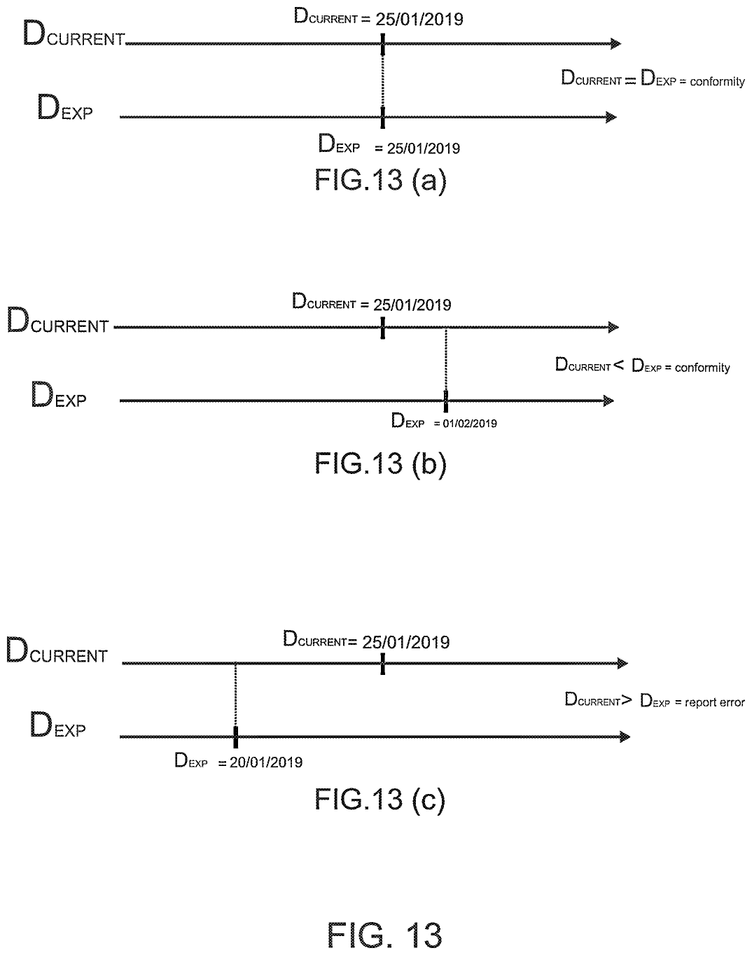

FIG. 13 is an illustration of the step of comparison between the expiration date D_{EXP} data and data referring to the current date D_{CURRENT}. In one example illustrated in FIG. 13 (a), the product identifier code was read on a current date D_{CURRENT} that is equal to the expiry date D_{EXP} of the product.

In this instance, comparison between the dates shows that they are the same, which, despite representing a limiting scenario, is still in a compliance status. Thus, in the scenario illustrated in FIG. 13(a), the step of reading the product identifier code is in a compliance status, as also represented in block B2 of FIG. 2.

The same applies to the scenario illustrated in FIG. 13 (b), where a comparison between the dates shows that the current date D_{CURRENT} is lower than (that is, prior to) the expiration date D_{EXP} of the product. In this instance, reading of the product identifier code is in a compliance status.

In such compliance instances, and upon reading the product identifier code 2, the first sound signal should be emitted, as mentioned above.

The comparison illustrated in FIG. 13 (c) shows that the current date D_{CURRENT} is greater (that is, later) than the expiration date D_{EXP} of the medical technology product. In this case, there is a situation of non-compliance (according to block B3 of FIG. 2), since the product 1 is in use after its expiration date.

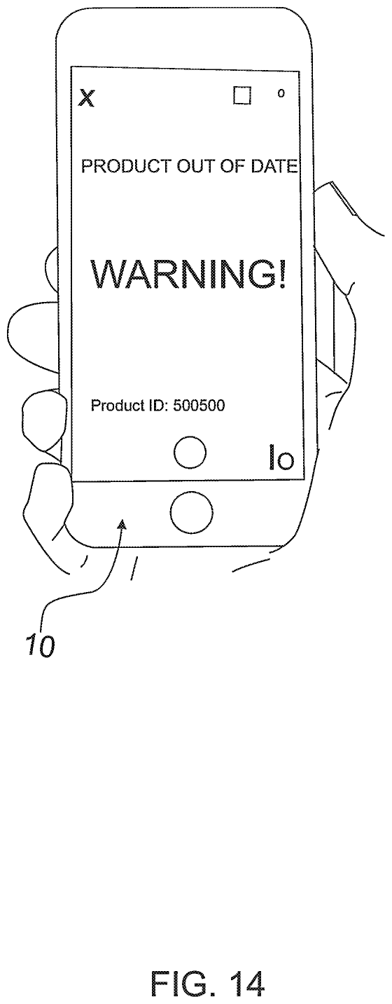

In this instance, the methodology proposed in the present invention proposes that an error message (alert signal) be reported to the health care setting operator, as indicated in block B4 of FIG. 2. More specifically, it is proposed that said error message be displayed on the screen of the first device 10, as illustrated in FIG. 14.

In addition, or alternatively to the emission of the error message, the second sound signal can be issued to the user, thus alerting him/her about the non-compliance between the current date D_{CURRENT} and the expiration date D_{EXP}.

As discussed above, the second sound signal is configured as a different sound than the first sound signal.

By sound signal is meant the emission of an audible sound to the operator of the first device 10 and/or to the health care setting manager.

Once this error message is displayed together with the sound signal, the health care setting operator will be aware that such medical technology product 1 should not be used. A warning to the health care setting manager can also be displayed in the second environment 20 if any collection using expired tubes took place, so the indicator to the manager I_{G} can be configured as a warning relative to the occurrence of collections using expired tubes. It is proposed that the sound signaling, preferably the second sound signaling, is also issued to the health care setting manager in a situation of non-compliance.

It is thus proposed that the step of reading the product identifier code further comprises the step of comparing the product expiration date D_{EXP} data 1 with the current date D_{CURRENT} data.

Thus, if such comparison shows that the data referring to the current date D_{CURRENT} is greater (that is, later) than the expiration date D_{EXP}, as illustrated in FIG. 13 (c), the methodology proposed in the present invention also teaches the step of displaying an error message on the screen of the first device 10, as shown in FIG. 14. By error message is meant an alert message to the operator showing that the medical technology product whose code 2 was read has expired. Said error message can be understood as a visual indicator to the health care setting operator I_{O}. Furthermore, a sound signal (audible indication) can also be issued to the operator, as previously described.

Moreover, if the comparison between D_{CURRENT} and D_{EXP} shows that these dates are the same (FIG. 13(a)) or shows that the current date D_{CURRENT} is prior to the expiration date D_{EXP} (FIG. 13(b)), a compliance status is in place. In this case, an error message is not displayed to the operator. Eventually, a compliance message may be displayed, but such a step should not be considered as a limiting feature of the present invention. As described above, an audible indication can be issued (first sound signal) to the operator, indicating the conformity between the dates.

Additionally, it is proposed that the health care setting operator who performs the methodology proposed in the present invention by the first device 10 can perform an initial step (check in) and a final step (check out) using such first device 10. By initial step is meant the beginning of the procedures to be performed by the operator, whereas the final step is meant as the end of the procedures performed by the operator. The initial step can be understood as the beginning of a working day, while the final step can be understood as the end of a working day.

In connection with the previously provided description, the present invention further describes a system capable of carrying out the previously described methodology.

Thus, a system for tracking a medical technology product is proposed, wherein the system comprises at least one medical technology product 1 provided with an identifier code 2 and further comprising the first device 10 able to read the product identifier code 2. In accordance with the previously made description, the reading of the product identifier code 2 is performed by using the optical instrument (camera) of the first device 10 or an optical instrument that is associated with the first device 10.

As previously described, the system further comprises means for linking the product identifier code 2 to the patient's identifier code 4, thus generating the consolidated data D_{C}.

In one embodiment, the consolidated data D_{C} can be generated by a microcontroller and/or processor associated with the first device 10 or by a microcontroller and/or processor associated with the second device 20. The microcontroller and/or processor may be arranged on the first and second devices, or can be arranged in an environment remote from such devices, such as a cloud environment.

In accordance with the previously made description, the system proposed in the present invention further comprises means to generate a plurality of indicators I_{O} and I_{G}, such as an indicator to the health care setting operator I_{O} and an indicator to the health care setting manager I_{G}. Such indicators can be generated by the microcontroller and/or processor of the first device 10 and/or the second device 20, as previously described.

As previously described, the indicator to the health care setting operator I_{O} is displayed to the health care setting operator, and more specifically on the screen of the first device 10.

The indicator to the health care setting manager I_{G}, on the other hand, and in accordance with the previously made description, is displayed to the health care setting manager, and more specifically on the screen of the second environment 20 (second device 20).

Thus, the system proposed in the present invention further comprises a second device 20, which device should be understood as a web platform that can be accessed by the health care setting manager.

It is proposed that the first device 10 and the second device 20 may exchange data with each other, such as the consolidated data D_{C} generated from the link between the medical technology product and the patient.

It is also proposed that the first device 10 and the second device can store and process data, such as the consolidated data D_{C} generated from the link between the medical technology product and the patient.

Accordingly, and in accordance with the previously provided description, the system proposed in the present invention is set to display a collection sequence S_{C} on the screen of the first device, as illustrated in FIGS. 7, 8 and 9. Moreover, the system proposed in the present invention is further configured to display on the screen of the second device 20, at least one of: an activity report R_{A}, a test panel P_{E} and a control panel P_{C} as respectively illustrated in FIGS. 10, 11 and 12.

As previously described, the system proposed in the present invention is also configured to compare a expiration date D_{EXP} data with a current date D_{CURRENT} data, thus assessing whether the current date D_{CURRENT} is equal to, greater than or less than the expiration date D_{EXP}, as illustrated in FIG. 13.

Thus, the system proposed in the present invention is also set to display on the screen of the first device 10 an error message if the comparison between the expiration date D_{EXP} data and the current date D_{CURRENT} data reports a non-compliance scenario, as illustrated in FIGS. 13 and 14.

Furthermore, the system proposed in the present invention is also set to issue at least a first sound signal and a second sound signal, as previously described.

In summary, the system proposed in the present invention can carry out the previously described tracking methodology.

The present invention further describes a non-transient computer-readable medium, the readable medium storing instructions, which instructions when executed by a processor carry out the steps of: linking a product identifier code to a patient's identifier code, and generating a plurality of indicators from the step of linking the product identifier code to a patient's identifier code. It is understood that the readable medium can perform any of the steps that are part of the methodology proposed in the present invention.

The present invention further describes a memory capable of storing and processing any of the steps described in the proposed methodology.

A medical technology product 1,160 is also described, which is provided with a product identifier code 2, the medical technology product 1,160 being handled by a heath care setting operator, wherein handling of medical technology product 1 allows the product identifier code be linked to a patient's identifier code C, generating a plurality of indicators I_{O}, I_{G} from said link between the product identifier code 2 and the patient's identifier code 4.

It is understood that the handling of medical technology product 1,160 by the health care setting operator allows any of the steps described in the present invention be carried out.

Thus, a method and system are proposed to allow the tracking, control and management of a medical technology product used in a heath care setting. Thus, the invention allows tubes used in hospital settings to be tracked, such as the tracking of tubes that are supplied to/from a support laboratory. By support laboratory is meant a laboratory that receives/supplies medical technology product from/to a main laboratory.

Furthermore, the teachings of the present invention also allow for the tracking, control and management of a set of medical technology products, such as a set of products that are arranged in a particular enclosure (such as a box or package) and that is received by a health care setting. Thus, the term medical technology product can be interpreted as a single medical technology product, as a set of medical technology products or even as a single medical technology product that stores a set of medical technology product.

Thus, and with reference to FIG. 16, the teachings of the present invention allow tracking of a medical technology product 160, such as a package that stores a set of collection tubes and/or needles.

In this instance, and as previously described, association of the medical technology product 160 with the product identifier code 2 is proposed, so that, for such an association to occur, an adhesive means 30 could be used. Said adhesive means 30 may comprise a portion P which acts as a criticality classifier, as previously described.

Thus, one can carry out the tracking of medical technology product 160, from a point of origin (manufacturer) to a point of destination (receiver), also evaluating whether the medical technology product 160 has effectively left the origin and reached its destination.

To this end, it is suggested that in the origin a dispatch reading Lo of the product identifier code 2 is made, thereby identifying the data (information) present in such code 2.

When arriving at the destination point, a new reading of the product identifier code 2 is proposed, such reading being referenced to as receipt reading L_{R}.

Thus, when receiving the material at the point of origin, the step of receipt reading L_{R} allows the receiver to evaluate the information/data present in the product identifier code 2, also evaluating integrity of the package 160 received as well as the medical technology products 1 arranged therein.

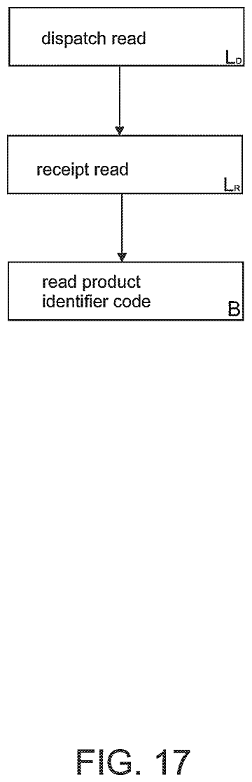

Accordingly, the steps of dispatch reading Lo and receipt reading L_{R} act as confirmation steps that the medical technology product has actually left its origin and arrived at its destination (thus ensuring the chain of custody in the transportation). Furthermore, and with reference to FIGS. 2 and 17, after performing the receipt reading L_{R}, the step of reading the product identifier code B can be performed, as previously described.

It is also understood that the steps of dispatch reading Lo and receipt reading L_{R} are part of the previously described method of tracking a medical technology product, as well as the system connected to such a method.

Also, the terms point of origin and point of destination should be understood as any location to which a specific medical technology product 1,160 is to be sent, that is, it is understood that the medical technology product 1,160 must be sent from the origin to a destination point.

Thus, in a non-limiting embodiment, the origin can be understood as the product 1,160 manufacturer (such as the manufacturer of a collection tube or a set of needles), whereas the destination can be understood as the laboratory receiving the product 1,160.

In an equally valid embodiment, the points of origin and destination can be understood as different locations within the same health care setting, or even in different health care settings.

Thus, the teachings of the present invention allow a certain medical technology product 1,160 to be tracked from an origin to its destination; to this end, the medical technology product should comprise an identifier code 2, as previously described.

Further, the product identifier code 2 and/or the patient's identifier code may be a unique identifier code. Additionally, the plurality of indicators I_{O}, I_{G} is and/or represent said unique identifier code.

Having described a preferred embodiment of the present invention, it should be understood that the scope of the present invention encompasses other possible variations, being limited only by the content of the appended claims, including any possible equivalents thereof.

1. A method of tracking medical technology product (100), the medical technology product (1,160) being provided with a product identifier code (2), the method comprising the steps of:
linking the product identifier code to a patient's identifier code (C), and
generating a plurality of indicators (I_{O}, I_{G}) from the step of linking the product identifier code (2) to a patient's identifier code (4).

2. The method (100) according to claim 1, wherein the indicators are associated with at least one of:
an operator of a health care setting, or
a manager of the health care setting.

3. The method (100) according to claim 2, wherein the plurality of indicators (I_{O},I_{G}) comprises at least one indicator to the health care setting operator (I_{O}), wherein the indicator to the health care setting operator (I_{O}) is displayed to the health care setting operator.

4. The method (100), according to claim 3, wherein the indicator to the health care setting operator (I_{O}) correlates a plurality of medical technology products (1) with a plurality of users of the health care setting.

5. The method (100), according to claim 2, wherein the plurality of indicators (I_{O}, I_{G}) comprises at least one indicator to the health care setting manager (I_{G}), wherein the indicator to the health care setting manager (I_{G}) is displayed to the manager of the health care setting.

6. The method (100), according to claim 1, wherein the indicator to the health care setting operator (I_{O}) is displayed in a first environment (10) and the indicator to the health care setting manager (I_{G}) is displayed in a second environment (20), wherein the plurality of indicators (I_{O}, I_{G}) at least one of is or represent a unique identifier code.

7. The method (100), according to claim 1, wherein the product identifier code (2) is associated with the medical technology product (1,160) through an adhesive means (30), wherein at least one portion (P) of the adhesive means (30) acts as a criticality classifier, wherein the criticality classifier indicates a criticality level of at least one of the medical technology product (1,160) or a patient.

8. The method (100), according to claim 2, wherein the product identifier code (2) is an optical readable code.

9. The method (100), according to claim 1, wherein the step of linking the product identifier code to a patient's identifier code (C) further comprises the step of reading the product identifier code (B).

10. The method (100), according to claim 9, wherein the step of reading the product identifier code further comprises the step of comparing a medical technology product expiration date with a current date (B1).

11. The method (100) according to claim 10, wherein the step of comparing a medical technology product expiration date (D_{EXP}) with a current date (D_{CURRENT}) results in a status of compliance or a status of non-compliance, so that:
in the compliance status, a first sound signal is issued to the health care setting operator,
in the non-compliance status, a second sound signal is issued to the health care setting operator,
wherein the first sound signal is different from the second sound signal.

12. The method (100), according to claim 9, wherein the step of reading the product identifier code (B) is performed by an optical instrument.

13. The method (100), according to claim 1, wherein the step of linking the product identifier code to a patient's identifier code (C) is performed by the health care setting operator.

14. The method (100), according to claim 1, wherein the product identifier code (2) comprises at least one of:
a medical technology product description data (D_{DESCR}),
a medical technology product batch data, or
a medical technology product expiration date (D_{EXP}).

15. The method according to claim 1, further comprising the step of:
issuing an alert signal to the health care setting operator, wherein the alert signal is set as at least a visual indication and an audible indication.

16. The method (100) according to claim 1, further comprising the step of generating a collection sequence (S_{C}) to the health care setting operator and displaying the collection sequence (S_{C}) on the first device (10).

17. The method (100), according to claim 1, wherein the medical technology product (1) is one of the following materials:
a collection tube (1),
a vaccine, and
a needle.

18. The method (100) according to claim 1, further comprising the step of:
generating and displaying a test panel (P_{E}) to the health care setting manager, wherein the test panel (P_{E}) correlates data:
referring to the health care setting unit where the link was generated,
referring to the test performed (D_{TEST}), and
referring to the number of tests performed for a given health care setting unit (N_{TEST}).

19. The method (100) according to claim 1, further comprising the step of:
generating and displaying a control panel (P_{C}) to the health care setting manager,
wherein the control panel (P_{C}) is configured as a graphical indicator that correlates a specific period of time, the number of links generated and the type of medical technology product.

20. The method (100) according to claim 1, further comprising the steps of:
performing a dispatch reading (L_{D}) of the medical technology product (1,160), wherein the dispatch reading (L_{D}) is held at a point of origin, and
performing a receipt reading (L_{R}) of the medical technology product (1,160), wherein the receipt reading (L_{R}) is held at a destination point.

21. A system for tracking medical technology products, the system comprising:
at least one medical technology product (1,160) provided with an identifier code (2),
a first device (10) able to read the product identifier code (2),
means to link the product identifier code (2) to a patient's identifier code (4), and
means for generating a plurality of indicators (I_{O}, I_{G}) from the link between the product identifier code (2) and the patient's identifier code (4).

22. The system according to claim 21, wherein the plurality of indicators (I_{O},I_{G}) comprises at least one of:
an indicator to the health care setting operator (I_{O}), wherein the indicator to the health care setting operator is displayed to the health care setting operator, or
an indicator to the health care setting manager (I_{G}), wherein the indicator to the health care setting manager (I_{G}) is displayed to the health care setting manager,
wherein the indicator to the health care setting operator (I_{O}) is displayed in a first environment (10) and the indicator to the health care setting manager (I_{G}) is displayed in a second environment (20).

23. A non-transitory computer-readable medium, the readable medium being characterized in that it stores instructions, which instructions when executed by a processor perform the steps of:
linking a product identifier code to a patient's identifier code (C), and
generating a plurality of indicators (I_{O}, I_{G}) from the step of linking the product identifier code (2) to a patient's identifier code (4).

24. A computer readable memory containing instructions for the method as recited in claim 1.

25. A medical technology product (1,160), provided with a product identifier code (2), the medical technology product (1,160) being handled by a health care setting operator, wherein handling of medical technology product (1) allows:
linking the product identifier code to a patient's identifier code (C), and
generating a plurality of indicators (I_{G}, I_{G}) from the link between the product identifier code (2) and the patient's identifier code (4).
